(19) United States
(12) Patent Application Publication
(10) Pub. No.: US 2016/0287383 A1
(43) Pub. Date: Oct. 6, 2016

(54) HEART VALVE REPAIR DEVICES

(71) Applicant: Edwards Lifesciences Corporation, Irvine, CA (US)

(72) Inventor: Stanton J. Rowe, Newport Coast, CA (US)

(73) Assignee: Edwards Lifesciences Corporation, Irvine, CA (US)

(21) Appl. No.: 15/086,916

(22) Filed: Mar. 31, 2016

Related U.S. Application Data
(60) Provisional application No. 62/141,646, filed on Apr. 1, 2015.

(51) Int. Cl. A61F 2/24

(52) U.S. Cl. CPC A61F 2/243; A61F 2/2427

(57) ABSTRACT

In one representative embodiment, a heart valve repair device comprises a plurality of prongs which are configured to torque a native heart valve leaflet to create a fold in the native heart valve leaflet.

### CROSS REFERENCE TO RELATED APPLICATION

This application claims the benefit of U.S. Provisional Application Ser. No. 62/141,646, entitled HEART VALVE REPAIR DEVICES, filed on Apr. 1, 2015, which is incorporated by reference herein in its entirety.

### FIELD

The present disclosure generally relates to heart valve repair and more particularly to devices and related methods for repairing heart valve leaflets.

### BACKGROUND

The native heart valves (i.e., the aortic, pulmonary, tricuspid and mitral valves) serve critical functions in assuring the forward flow of an adequate supply of blood through the cardiovascular system. These heart valves can be rendered less effective by congenital malformations, inflammatory processes, infectious conditions, or disease. Such damage to the valves can result in serious cardiovascular compromise or death. For many years the definitive treatment for such disorders was the surgical repair or replacement of the valve during open heart surgery. However, such surgeries are highly invasive and are prone to many complications. Therefore, elderly and frail patients with defective heart valves often went untreated. More recently, transcatheter techniques have been developed for introducing and implanting prosthetic devices in a manner that is much less invasive than open heart surgery. Such transcatheter techniques have increased in popularity due to their high success rates.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps or leaflets extending downward from the annulus into the left ventricle. The mitral valve annulus can form a “D” shaped, oval, or otherwise out-of-round cross-sectional shape having major and minor axes. The anterior leaflet can be larger than the posterior leaflet, forming a generally “C” shaped boundary between the abutting free edges of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the two leaflets together, thereby closing the one-way mitral valve so that blood cannot flow back to the left atrium and is instead expelled out of the left ventricle through the aortic valve. To prevent the two leaflets from prolapsing under pressure and folding back through the mitral annulus toward the left atrium, a plurality of fibrous cords called chordae tendineae tether the leaflets to papillary muscles in the left ventricle.

Mitral regurgitation occurs when the native mitral valve fails to close properly and blood flows into the left atrium from the left ventricle during the systolic phase of heart contraction. Mitral regurgitation is the most common form of valvular heart disease. Although there are many different causes of mitral regurgitation, one particular cause is excessive slack in at least one of the native leaflets. This excessive slack prevents the native leaflets from effectively closing during the systolic phase of heart contraction, thus allowing mitral regurgitation.

Various devices and methods for treating mitral regurgitation have been developed, including implanting a prosthetic valve within the native mitral valve or surgically removing a portion of the native heart valve leaflets to reduce excessive slack. These devices and methods can, however, be highly invasive or require an extensive recovery period.

Thus, there is a continuing need for improved devices and methods for repairing native heart valve leaflets.

### SUMMARY

Described herein are embodiments of devices that are primarily intended to be used to repair the native leaflets of the mitral, aortic, tricuspid, or pulmonary heart valve, as well as methods for repairing the same. The devices can be used to remove excess slack in a native heart valve leaflet by folding the native leaflet.

In one representative embodiment, a heart valve repair device comprises a plurality of prongs which are configured to torque a native heart valve leaflet to create a fold in the native heart valve leaflet.

In some embodiments, at least one of the prongs is moveable or pivotable relative to the other prongs.

In some embodiments, the heart valve repair device further comprises a handle and an elongate shaft, and the handle is connected to a first end of the shaft and the plurality of prongs are connected to a second end of the shaft. In some embodiments, the shaft is configured such that torquing the handle in a first direction about a first axis causes plurality of prongs to rotate in the first direction about a second axis. In other embodiments, the shaft is configured such that torquing the handle in a first direction about a central axis causes plurality of prongs to rotate in the first direction about the central axis.

In some embodiments, the shaft comprises an inner shaft, the device further comprises an outer shaft, and the inner shaft extends co-axially through the handle and the outer shaft. In some embodiments, the inner shaft is fixedly secured to the handle and the plurality of prongs at opposite ends of the inner shaft.

In some embodiments, the prongs are configured to torque a free end of the native heart valve leaflet. In some embodiments, the prongs are laterally spaced apart such that the native heart valve leaflet can be positioned between the prongs.

In some embodiments, at least one prong has at least one ejection port which is configured to eject an adhesive. In some embodiments, the device is configured to eject a fastener.

In another representative embodiment, a method for repairing a heart valve is provided. The method for repairing a heart valve can comprise folding a native heart valve leaflet into a folded state by torquing a heart valve repair device.

In some embodiments, the method further comprises positioning a plurality of prongs of the heart valve repair device such that at least one of the prongs contacts a first side of the native heart valve leaflet and at least one of the prongs contacts a second side of the native heart valve leaflet.

In some embodiments, torquing a heart valve repair device comprises torquing a handle in a first direction about a first axis which causes the plurality of prongs to rotate in the first direction about a second axis. In other embodiments, torquing a heart valve repair device comprises torquing a handle in a first direction about a central axis which causes the plurality of prongs to rotate in the first direction about the central axis.

In some embodiments, the method further comprises securing the native heart valve leaflet in the folded state. In some embodiments, the native heart valve leaflet is secured in the folded state by injecting an adhesive into a space between a first and a second folded portion of the native heart valve leaflet. In some embodiments, the native heart valve leaflet is secured in the folded state by inserting a fastener into the native heart valve leaflet.

In some embodiments, folding a native heart valve leaflet includes folding a free end of the native heart valve leaflet.

In another representative embodiment, another method for repairing a heart valve is provided. The method for repairing a heart valve can comprise folding a native heart valve leaflet into a first folded state by pivoting or moving at least one prong of a heart valve repair device relative to at least one other prong of the heart valve repair device. The method can also comprise folding the native heart valve leaflet from the first folded state to a second folded state by torquing the heart valve repair device.

In some embodiments, the method further comprises securing the native heart valve leaflet in the first folded state prior to folding the native heart valve leaflet into the second folded state. In some embodiments, the method further comprises securing the native heart valve leaflet in the second folded state.

In some embodiments, securing the native heart valve leaflet in the first or second folded states includes applying an adhesive to the native heart valve leaflet. In some embodiments, securing the native heart valve leaflet in the first or second folded states includes inserting a fastener into the native heart valve leaflet.

The foregoing and other objects, features, and advantages of the invention will become more apparent from the following detailed description, which proceeds with reference to the accompanying figures.

### BRIEF DESCRIPTION OF THE DRAWINGS

FIG. 1A is a perspective view of a heart valve repair device, according to one embodiment.

FIG. 1B is a perspective view of the heart valve repair device of FIG. 1A inserted through the left atrium to the mitral position of a heart, which is shown in partial cross-section.

FIGS. 1C-1D show cross-sectional views of the heart valve repair device of FIG. 1A folding a native mitral valve leaflet into a folded state.

FIG. 1E shows the native mitral valve leaflet secured in the folded state of FIG. 1D after removal of the repair device.

FIG. 2A is a perspective view of a heart valve repair device, according to another embodiment.

FIGS. 2B-2C show cross-sectional views of the heart valve repair device of FIG. 2A folding a native mitral valve leaflet into a folded state.

FIG. 2D shows the native mitral valve leaflet secured in the folded state of FIG. 2C after removal of the repair device.

FIG. 2E shows the native mitral the heart valve repair device of FIG. 2A folding a native mitral valve leaflet into another folded state.

FIG. 2F shows the native mitral valve leaflet secured in the folded state of FIG. 2E after removal of the repair device.

### DETAILED DESCRIPTION

Described herein are embodiments of devices that are primarily intended to be used to repair the native leaflets of the mitral, aortic, tricuspid, or pulmonary heart valve, as well as methods for repairing the same. The devices can be used to remove excess slack in a native heart valve leaflet by folding the native leaflet. By removing the excessive slack in the native leaflet, these devices can reduce or improve valvular regurgitation and, thus, improve the functionality of a defective native heart valve.

In particular embodiments, a heart valve repair device can be configured to repair a native mitral valve leaflet. The device can access the mitral valve from the left ventricle and/or the left atrium in a minimally invasive manner (e.g., using a transcatheter technique).

In particular embodiments, the heart valve repair device includes a plurality of prongs or tines which can torqued or rotated to create a fold in a native mitral valve leaflet.

In particular embodiments, the heart valve repair device can also be used to secure the native leaflet in the folded state.

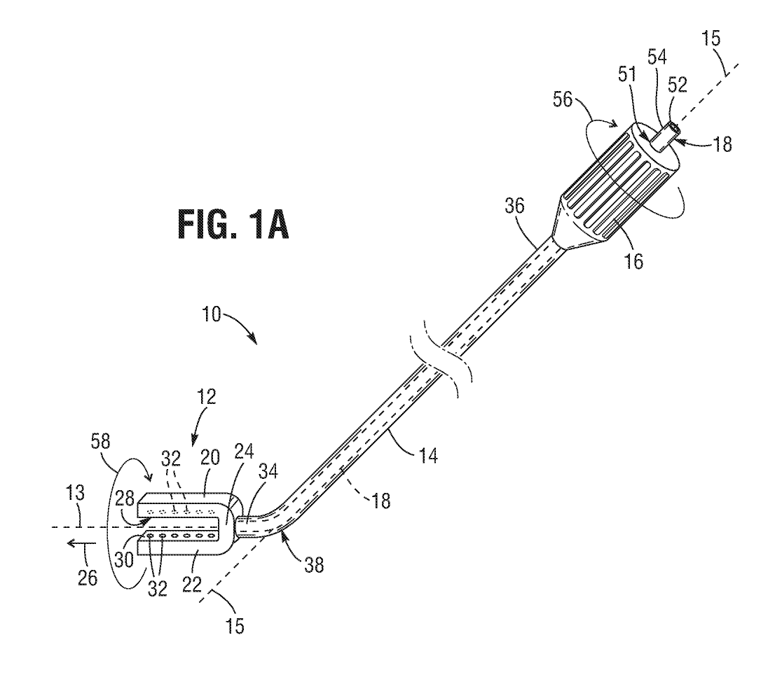

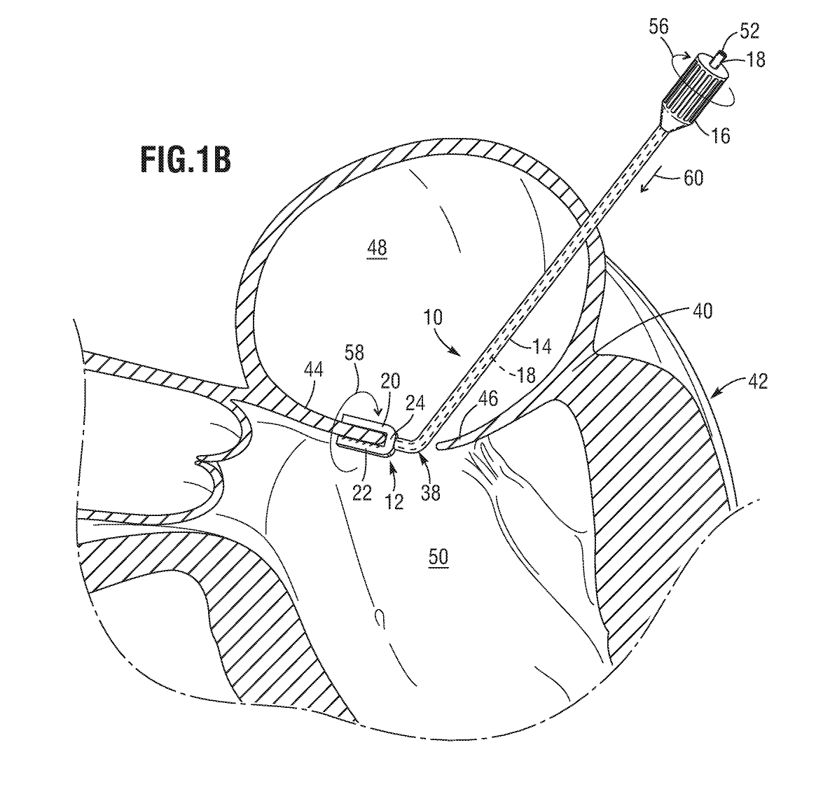

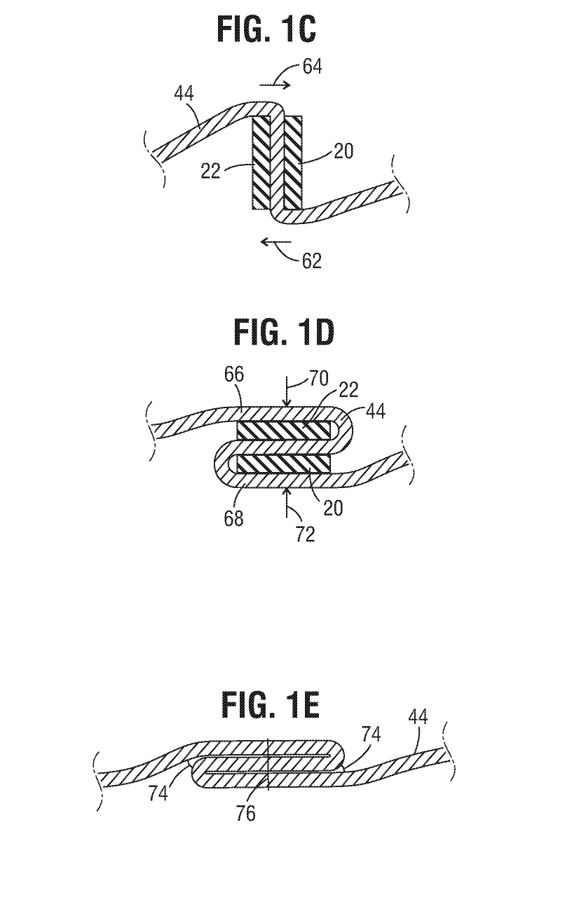

Referring first to FIG. 1A, there is shown a heart valve repair device 10 for folding a native heart valve leaflet (e.g., a native mitral valve leaflet), according to one embodiment. The device 10 comprises a distal portion 12, an outer shaft 14, a handle 16, and an inner shaft 18. The outer shaft 14 can be disposed between the distal portion 12 and the handle 16. The inner shaft 18 can extend co-axially through the handle 16 and the outer shaft 14 and can be connected at opposite ends to the distal portion 12 and the handle 16. The distal portion 12, inner shaft 18, and the handle 16 can be rotatable relative to the outer shaft 14, as further described below.

The repair devices described herein (e.g., devices 10, 100) are described in the context of repairing a native mitral valve leaflet. However, it should be understood that the repair devices can be used to repair leaflets of the other native heart valves.

The distal portion 12 can comprise a plurality of tines or prongs (two in the illustrated embodiment), including at least a first prong 20 and at least a second prong 22. The distal portion 12 can also comprise a connecting member 24 which can be fixedly secured to the inner shaft 18. The prongs 20, 22 can be fixedly secured or connected at their proximal ends to the connecting member 24.

As shown, the prongs 20, 22 can, for example, be spaced apart laterally and substantially parallel relative to each other as the prongs 20, 22 extend away from the connecting member 24 in the distal direction (i.e., the direction shown by arrow 26). The lateral spacing between the prongs can be such that a native heart valve leaflet can be positioned between the first prong 20 and the second prong 22, as best shown in FIG. 1B. Configuring the prongs 20, 22 in this manner allows the prongs 20, 22 to capture and fold a native mitral valve leaflet, as further described below.

In the illustrated embodiment, the distal portion 12 comprises a single, unitary “U”-shaped component defining the prongs 20, 22 and the connecting member 24. In alternative embodiments, the prongs 20, 22 and the connecting member 24 can be formed from separate pieces of material which are fixedly secured or coupled together by, for example, an adhesive, welding, fasteners, etc.

The prongs 20, 22 can, for example, each comprise a generally rectangular cross-sectional shape, as best shown in FIG. 1D. It should be noted that the prongs 20, 22 can comprise various cross-sectional shapes (e.g., circular, elliptical, etc.).

As shown in FIG. 1A, the respective prongs 20, 22 can, for example, comprise a plurality of ejection ports 32 disposed on the inner surfaces 28, 30 of the prongs (i.e., the surfaces that face each other), respectively. The ejection ports 32 can, for example, be configured to release or eject a biocompatible adhesive (e.g., BioGlue® manufactured by CryoLife, Inc.) onto a native leaflet. The adhesive can be used to secure a native leaflet in a folded state, as further described below.

The outer shaft 14 of the device 10 can be disposed between the connecting member 24 and the handle 16 such that a distal end 34 of the outer shaft 14 is adjacent to and/or abutting the connecting member 24 and a proximal end 36 of the outer shaft 14 is adjacent to and/or abutting the handle 16. In an alternative embodiment, the outer shaft 14 can be rotatably connected to the connecting member 24 and the handle 16 at opposite ends such that the connecting member 24 and the handle 16 are rotatable relative to the outer shaft 14. This can be accomplished, for example, by connecting the outer shaft 14 to the distal portion 12 and the handle 16 with respective bearings.

The outer shaft 14 can, for example, be configured to assist in positioning and operating the device 10 based on the particular transcatheter approach used to access the native mitral valve and/or which native leaflet is being repaired. For example, in the illustrated embodiment, the outer shaft 14 comprises a bent or curved portion 38 located near the distal end 34 of the outer shaft 14, as best shown in FIG. 1A. Configuring the outer shaft 14 in this manner can, for example, assist in the positioning and operating the device 10 when accessing a native mitral valve 40 of a heart 42 using a transatrial approach, as shown in FIG. 1B. Specifically, the curved portion 38 of the outer shaft 14 allows the distal portion 12 to capture the native anterior leaflet 44 without substantially interfering with the posterior leaflet 46, left atrium 48, left ventricle 50, and/or other anatomy of the heart 42, as shown in FIG. 1B. The curved portion 38 also positions the handle 16 in a location accessible to a physician operating the device 10.

The handle 16 can comprise an axially extending lumen having a proximal opening 51 (FIG. 1A) which can allow other components (e.g., the inner shaft 18) to extend co-axially through the handle 16. The handle 16 can be fixedly secured to the inner shaft 18 such that rotating the handle 16 causes the inner shaft 18 to rotate, as further described below.

The inner shaft 18 can comprise an injection port 52 disposed at a proximal end 54 of the inner shaft 18, as best shown in FIG. 1A. The injection port 52 of the inner shaft 18 can be configured to be in fluidic communication with the ejection ports 32 of the distal portion 12. In this manner, a fluid (e.g., BioGlue) can flow into the injection port 52 and flow out of the ejection ports 32 of the prongs 20, 22, as further described below.

The device 10 can be configured such that torquing the handle 16 in a first direction causes the distal portion 12 to rotate in the same direction about its respective central axis 13, and torquing the handle 16 in a second direction, opposite the first, causes the distal portion 12 to rotate in the opposite direction about its respective central axis 13. For example, as shown in FIG. 1A, torquing the handle 16 in the direction of arrow 56 (i.e., clockwise in the illustrated embodiment) causes the inner shaft 18 and thus the distal portion 12 to rotate clockwise in the direction shown by arrow 58 about axis 13. Conversely, torquing the handle 16 counterclockwise causes the inner shaft 18 and thus the distal portion 12 to rotate in the counterclockwise direction about axis 13. This can be accomplished, for example, by forming the inner shaft 18 from a flexible material such as a braided or twisted wire, similar to a flexible socket extension.

In an alternative embodiment, for example, the inner shaft 18 can further comprise a mechanism (e.g., a u-joint or cv-joint, etc.) disposed within the outer shaft 14 that allows the handle 16 and the distal portion 12 to simultaneously rotate about their respective axes 15, 13, respectively.

In another alternative embodiment, the device 10 can be configured such that torquing the handle 16 in a first direction causes the distal portion 12 to rotate in the same direction about a central axis 15 of the handle 16, and torquing the handle 16 in a second direction, opposite the first, causes the distal portion 12 to rotate in the opposite direction about the central axis 15. This can be accomplished, for example, by rigidly connecting the distal portion 12, the outer shaft 14, and the handle 16.

The device 10 can, for example, be used to reduce or improve valvular regurgitation by folding a native heart valve leaflet. Folding the native leaflet can reduce or remove the slack in the native leaflet, which, in turn, allows the leaflet to create a more effective seal during ventricular systole. For example, FIGS. 1B-1E show the device 10 being used to fold the native anterior leaflet 44 of the mitral valve 40.

When using the device 10 to fold the native anterior leaflet 44, the device 10 can, for example, be inserted into the left atrium 48 through an opening in the left atrial wall of the heart 42, as shown in FIG. 1B. Although not shown, it will be appreciated by those of ordinary skill that an introducer and/or catheter can be used to access the left atrium 48, and the device 10 can be inserted into left atrium 48 through a lumen of the introducer and/or catheter.

It should be noted that the positioning of the disclosed devices (e.g., devices 10, 100) can be confirmed visually using imaging modalities such as fluoroscopy, X-ray, CT, and MR imaging. Echocardiography in either 2D or 3D can also be used to help guide the positioning of the devices.

Referring still to FIG. 1B, with the device 10 in the left atrium 48, the device 10 can be advanced to the mitral valve 40 (e.g., by advancing the handle 16 relative to the heart 42 in the direction of arrow 60). The device 10 can capture or receive the free end of native anterior leaflet 44, for example, by advancing the first prong 20 over (i.e., on the atrial side) the anterior leaflet 44 and the second prong 22 below (i.e., on the ventricular side) the anterior leaflet 44.

Once the leaflet 44 is captured between the prongs 20, 22, the leaflet 44 can be folded. The leaflet 44 can be folded, for example, by rotating or torquing the handle 16 in the direction of arrow 56 which causes the distal portion 12 to rotate in the direction of arrow 58. This can be accomplished, for example, by grasping the outer shaft 14 with one hand while grasping and rotating the handle 16 with another hand.

Referring now to FIGS. 1B-1D, as the distal portion 12 rotates in the direction of arrow 58 with the leaflet 44 captured between the prongs 20, 22, the first prong 20 forces the leaflet 44 in the direction shown by arrow 62, and the second prong 22 forces the leaflet 44 in the opposite direction, as shown by arrow 64. FIG. 1C shows the prongs 20, 22 and the leaflet 44 after the distal portion 12 rotates 90 degrees relative to the initial positioning of the distal portion 12 shown in FIG. 1B. FIG. 1D shows the prongs 20, 22 and the leaflet 44 after the distal portion 12 rotates 180 degrees in the direction of arrow 58 relative to the initial positioning of the distal portion shown in FIG. 1B (90 degrees in the direction of arrow 58 relative to the positioning of the distal portion 12 shown in FIG. 1C).

With the leaflet 44 in a folded state, for example, as shown in FIG. 1D, the leaflet 44 can be secured in the folded state. The leaflet 44 can be secured in the folded state, for example, by applying an adhesive 74 (e.g., BioGlue®) onto the leaflet 44. The adhesive 74 can be configured to adhere the folded portions of the leaflet together, as shown in FIG. 1E.

The adhesive 74 can be applied to the leaflet, for example, by injecting the adhesive 74 into the injection port 52 of the inner shaft 18, which then flows through the device 10, out of the ejection ports 32 of the prongs 20, 22, and into the spaces between the folded portions of the leaflet 44. Once the adhesive 74 is applied to the leaflet 44, the prongs 20, 22 can be retracted from the leaflet 44, and the adhesive 74 can hold or retain the leaflet 44 in the folded state, as shown in FIG. 1E.

In lieu of or in addition to using an adhesive, the leaflet 44 can be secured in the folded state, for example, by using a fastener (e.g., staples and/or sutures). For example, in FIG. 1E, the leaflet 44 is secured with the adhesive 74 and a suture 76. The device 10 can be configured to insert the fastener, or the device 10 can be used in conjunction with a separate fastener device.

Although not shown, the device 10 can have a clamping mechanism which can be configured to apply a compressive force on a first fold 66 and a second fold 68 of the folded portion of the leaflet 44 in the direction of arrows 70 and 72 (FIG. 1D) as the prongs are retracted from the leaflet. The clamping mechanism can assist in retaining the leaflet in the folded state and/or compress the folded portion of the leaflet into a thinner profile as the prongs are retracted. Alternatively, a separate clamping device (not shown) can be used to assist in retaining the leaflet in the folded state and/or compress the folded portion of the leaflet into a thinner profile as the prongs are retracted.

If additional slack needs to be removed, the device 10 can then be repositioned on another portion of the leaflet 44, and the procedure described above can be repeated. Once the desired amount of slack is removed from the leaflet 44, the device 10 can be removed from the patient's body by retracting the device 10 in the direction opposite the direction shown by arrow 60 (FIG. 1B).

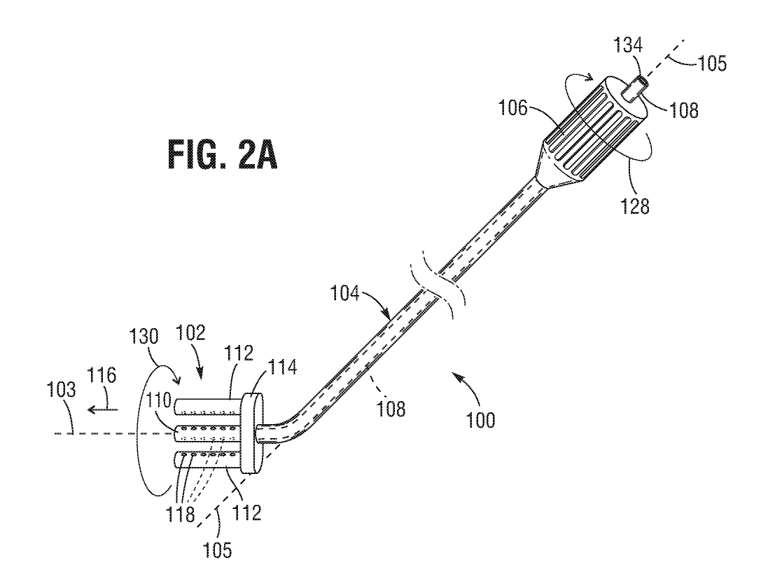

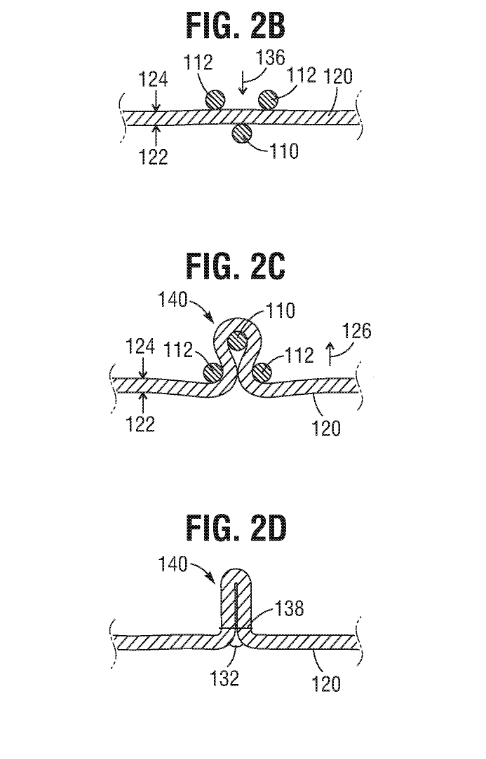

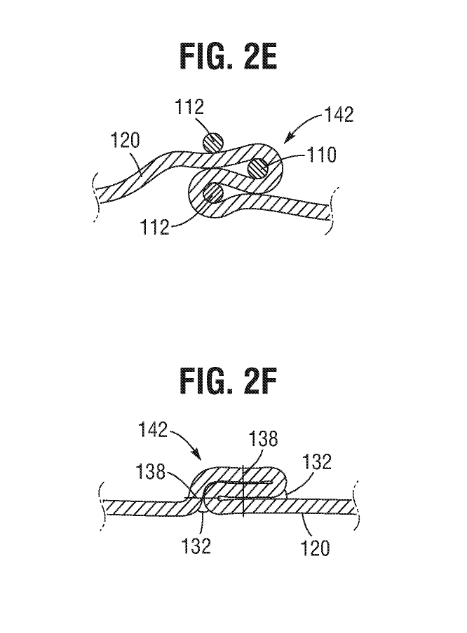

FIG. 2A shows a heart valve repair device 100 for folding a native heart valve leaflet (e.g., a native mitral valve leaflet), according to another embodiment. The device 100 comprises a distal portion 102, an outer shaft 104, a handle 106, and an inner shaft 108. The outer shaft 104 can be disposed between the distal portion 102 and the handle 106. The inner shaft 108 can extend co-axially through the handle 106 and the outer shaft 104 and can be connected at opposite ends to the distal portion 102 and the handle 106. The distal portion 102, inner shaft 108, and the handle 106 can be rotatable relative to the outer shaft 104, as further described below.

The distal portion 102 can comprise a plurality of tines or prongs (three in the illustrated embodiment), including at least one inner prong 110 and at least two outer prongs 112. The distal portion 102 can also comprise a connecting member 114 which can be fixedly secured to the inner shaft 108. The prongs 110, 112 can be connected to the connecting member 114 at their proximal ends.

As best shown in FIG. 2A, the prongs 110, 112 can be spaced apart laterally and configured such that the outer prongs 112 are positioned laterally outward from the inner prong 112. The prongs 110, 112 can be substantially parallel relative to each other as the prongs 110, 112 extend away from the connecting member 114 in the distal direction (i.e., the direction shown by arrow 116).

The inner prong 110 can be configured to be moveable and/or pivotable relative to the outer prongs 112, or vice versa. For example, the inner prong 110 can be configured to move from being aligned in a row (i.e., vertically in FIG. 2A) with the outer prongs 112 to being misaligned (i.e., horizontally in FIGS. 2B-2C) with the outer prongs 112. When configured in this manner, the prongs 110, 112 can, for example, be used to capture and fold a native mitral valve leaflet into a folded state by moving the inner prong 110 relative to the outer prongs 112, as further described below.

The prongs 110, 112 can, for example, each comprise a generally circular cross-sectional shape. However, it should be noted that the prongs 110, 112 can comprise various cross-sectional shapes (e.g., rectangular, elliptical, etc.).

As shown in FIG. 2A, the respective prongs 110, 112 can comprise a plurality of ejection ports 118, similar to ejection ports 32 of device 10. These ejection ports 118 can, for example, be configured to release or eject a biocompatible adhesive (e.g., BioGlue®) to secure a native leaflet in a folded state, as further described below.

The outer shaft 104, handle 106, and inner shaft 108 of device 100 can, for example, be configured in a manner substantially similar to the outer shaft 14, handle 16, and inner shaft 18 of device 10. Thus, in some embodiments, torquing the handle 106 clockwise or counterclockwise causes corresponding clockwise or counterclockwise rotation of the distal portion 102 about its respective central axis 103. In alternative embodiments, torquing the handle 106 clockwise or counterclockwise causes corresponding clockwise or counterclockwise rotation of the distal portion 102 about a central axis 105 of the handle 106.

The device 100 can be used to reduce or improve valvular regurgitation by folding a native heart valve leaflet. Folding the native leaflet can reduce or remove the slack in the native leaflet, which in turn promotes coaptation with the opposing leaflet or leaflets, thereby improving valve function. FIGS. 2B-2F show the device 100 being used to fold a native mitral valve leaflet 120.

When using the device 100 to fold a native mitral valve leaflet 120, the device 100 can, for example, be inserted into a heart and advanced to the mitral position in a manner similar to device 10, as described above. The device 100 can create a fold in the native leaflet 120 by capturing the leaflet 120 with the prongs 110, 112 and then by using the prongs to fold the leaflet, as further described below.

The device 100 can capture the free end of leaflet 120, for example, by orienting the prongs 110, 112 relative to the leaflet 120 such that the distal ends of the prongs 110, 112 are adjacent and substantially parallel to a free end of the leaflet 120. The inner prong 110 and/or the outer prongs 112 can be moved laterally relative to each other so as to create a space for the leaflet 120 between the prongs 110, 112. This can be accomplished, for example, by moving the inner prong 110 toward the ventricular side 122 of leaflet 120 (i.e., in the direction shown by arrow 136 in FIG. 2B), relative to the outer prongs 112, or vice versa. The distal portion 102 of the device 100 can then be positioned such that the free end of the leaflet 120 laterally aligns with the space between the inner and outer prongs 110, 112. The device 100 can then be advanced distally (i.e., in the direction of arrow 116 in FIG. 2A) relative to the leaflet 120. Advancing the device 100 in this manner moves the prongs 110, 112 over the free end of the leaflet 120 such that the inner prong 110 is on the ventricular side 122 of the leaflet 120 and the outer prongs 112 are on the atrial side 124 of the leaflet 120 (as shown in FIG. 2B), or vice versa.

With the leaflet 120 positioned between the prongs 110, 112, the leaflet 120 can be folded, for example, by torquing the handle 106 in the direction shown by arrow 128 (FIG. 2A) which in turn causes the distal portion 102 to rotate in the direction shown by arrow 130 (FIG. 2A) about its respective axis 103, in a manner to device 10 as described above.

Alternatively, the leaflet 120 can be folded or pleated, for example, by moving the inner prong 110 relative to the outer prongs 112 and leaflet 120, or vice versa. In the illustrated embodiment, for example, the leaflet 120 is folded by moving the inner prong 110 laterally toward the atrial side 124 of the leaflet (i.e., in the direction shown by arrow 126 in FIG. 2C) while maintaining the positioning of the outer prongs 112 relative to the leaflet 120. As the inner prong 110 moves laterally toward the atrial side 124 of the leaflet 120 such that the inner prong 110 laterally aligns with and moves laterally past the outer prongs 112, a portion of the leaflet 120 is forced between the outer prongs 112, thereby creating a fold or pleat in the leaflet 120, as shown in FIG. 2C.

The portion of the leaflet 120 that is folded can be adjusted by varying the positioning of the inner prong 110 relative to the outer prongs 112. For example, moving the inner prong 110 further toward the atrial side 124 of the leaflet 120 while maintaining the positioning of the outer prongs 112 forces a greater portion of the leaflet 120 between the outer prongs 112, thus increasing the portion of the leaflet 120 that is folded. On the other hand, moving the inner prong 110 toward the ventricular side 122 of the leaflet 120 while maintaining the positioning of the outer prongs 112 releases the portion of the leaflet 120 that is forced between the outer prongs 112, thus decreasing the portion of the leaflet 120 that is folded. Folding a greater portion of the leaflet removes more slack from the leaflet, while folding a lesser portion of the leaflet removes less slack from the leaflet.

Additionally, the leaflet 120 can be further folded from a first folded state 140 (FIG. 2C) to a second folded state 142 (FIG. 2E) by torquing the handle 106 and thus rotating the distal portion 102 in the direction of arrows 128, 130 (FIG. 2A), respectively. This torquing causes the leaflet 120 to fold over onto itself and gathers or pulls an additional portion of the leaflet 120 into the folded state 142, as shown in FIG. 2E.

Once the desired folded state (e.g., folded state 140 or 142) is achieved (e.g., as shown in FIGS. 2C or 2E), the leaflet 120 can be secured in the folded state such as by applying an adhesive, staples, and/or sutures to the leaflet 120, as described above. An adhesive 132 can be applied to the leaflet, for example, by injecting the adhesive 132 into an injection port 134 of the inner shaft 108, which can then flow through the device 100, out of the ejection ports 118 of the prongs 110, 112, and into the spaces between the folds of the leaflet 120. In the illustrated embodiment, for example, the leaflet 120 is secured in the various folded states by an adhesive 132 and sutures 138, as shown in FIGS. 2D and 2F.

With the leaflet 120 securely folded, the prongs 110, 112 can be retracted from the leaflet 120. Although not shown, the device 100 can have a clamping mechanism or a separate clamping device that can assist in retaining the leaflet in the folded state and/or compress the folded portion of the leaflet into a thinner profile as the prongs are retracted.

If additional slack needs to be removed, the device 100 can then be repositioned on another portion of the leaflet 120, and the procedure can be repeated. Once the desired amount of slack is removed from the leaflet 120, the device 100 can be removed from the patient's body.

It should be noted that the devices described herein (e.g., devices 10, 100) can be used with a various transcatheter techniques (e.g., transatrial, transventricular, etc.).

### General Considerations

For purposes of this description, certain aspects, advantages, and novel features of the embodiments of this disclosure are described herein. The disclosed methods, apparatuses, and systems should not be construed as limiting in any way. Instead, the present disclosure is directed toward all novel and nonobvious features and aspects of the various disclosed embodiments, alone and in various combinations and sub-combinations with one another. The methods, apparatuses, and systems are not limited to any specific aspect or feature or combination thereof, nor do the disclosed embodiments require that any one or more specific advantages be present or problems be solved.

Although the operations of some of the disclosed methods are described in a particular, sequential order for convenient presentation, it should be understood that this manner of description encompasses rearrangement, unless a particular ordering is required by specific language. For example, operations described sequentially may in some cases be rearranged or performed concurrently. Moreover, for the sake of simplicity, the attached figures may not show the various ways in which the disclosed methods can be used in conjunction with other methods.

As used herein, the terms “a”, “an” and “at least one” encompass one or more of the specified element. That is, if two of a particular element are present, one of these elements is also present and thus “an” element is present. The terms “a plurality of” and “plural” mean two or more of the specified element.

As used herein, the term “and/or” used between the last two of a list of elements means any one or more of the listed elements. For example, the phrase “A, B, and/or C” means “A,” “B,” “C,” “A and B,” “A and C,” “B and C” or “A, B and C.”

As used herein, the term “coupled” generally means physically coupled or linked and does not exclude the presence of intermediate elements between the coupled items absent specific contrary language.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. I therefore claim as my invention all that comes within the scope and spirit of these claims.

I claim:

1. A device for repairing a heart valve comprising:
a plurality of prongs, wherein the plurality of prongs are configured to torque a native heart valve leaflet to create a fold in the native heart valve leaflet.

2. The device of claim 1, wherein at least one of the prongs is moveable or pivotable relative to the other prongs.

3. The device of claim 1, further comprising a handle and an elongate shaft, wherein the handle is connected to a first end of the shaft and the plurality of prongs are connected to a second end of the shaft.

4. The device of claim 3, wherein the shaft is configured such that torquing the handle in a first direction about a first axis causes plurality of prongs to rotate in the first direction about a second axis.

5. The device of claim 3, wherein the shaft is configured such that torquing the handle in a first direction about a central axis causes plurality of prongs to rotate in the first direction about the central axis.

6. The device of claim 3, wherein the shaft comprises an inner shaft, the device further comprises an outer shaft, and the inner shaft extends co-axially through the handle and the outer shaft.

7. The device of claim 6, wherein the inner shaft is fixedly secured to the handle and the plurality of prongs at opposite ends of the inner shaft.

8. The device of claim 1, wherein the prongs are configured to torque a free end of the native heart valve leaflet.

9. The device of claim 1, wherein at least one prong has at least one ejection port which is configured to eject an adhesive.

10. The device of claim 1, wherein the device is configured to eject a fastener.

11. The device of claim 1, wherein the prongs are laterally spaced apart such that the native heart valve leaflet can be positioned between the prongs.

12. A method for repairing a heart valve comprising folding a native heart valve leaflet into a folded state by torquing a heart valve repair device.

13. The method of claim 12, further comprising positioning a plurality of prongs of the heart valve repair device such that at least one of the prongs contacts a first side of the native heart valve leaflet and at least one of the prongs contacts a second side of the native heart valve leaflet.

14. The method of claim 13, wherein torquing a heart valve repair device comprises torquing a handle in a first direction about a first axis which causes the plurality of prongs to rotate in the first direction about a second axis.

15. The method of claim 13, wherein torquing a heart valve repair device comprises torquing a handle in a first direction about a central axis which causes the plurality of prongs to rotate in the first direction about the central axis.

16. The method of claim 12, further comprising securing the native heart valve leaflet in the folded state.

17. The method of claim 16, wherein the native heart valve leaflet is secured in the folded state by injecting an adhesive into a space between a first and a second folded portion of the native heart valve leaflet.

18. The method of claim 16, wherein the native heart valve leaflet is secured in the folded state by inserting a fastener into the native heart valve leaflet.

19. The method of claim 12, wherein folding a native heart valve leaflet includes folding a free end of the native heart valve leaflet.

20. A method for repairing a heart valve comprising:
folding a native heart valve leaflet into a first folded state by pivoting or moving at least one prong of a heart valve repair device relative to at least one other prong of the heart valve repair device; and
folding the native heart valve leaflet from the first folded state to a second folded state by torquing the heart valve repair device.

21. The method of claim 20, further comprising securing the native heart valve leaflet in the first folded state prior to folding the native heart valve leaflet into the second folded state.

22. The method of claim 20, further comprising securing the native heart valve leaflet in the second folded state.

23. The method of claim 21, wherein securing the native heart valve leaflet in the first or second folded states includes applying an adhesive to the native heart valve leaflet.

24. The method of claim 21, wherein securing the native heart valve leaflet in the first or second folded states includes inserting a fastener into the native heart valve leaflet.
